(19) United States
(12) Patent Application Publication
(10) Pub. No.: US 2010/0179374 A1
(43) Pub. Date: Jul. 15, 2010

(54) Method and Apparatus for Treating a Heat Condition

(75) Inventor: Anthony Clyde Neason Stephens, Castlecrag New South Wales (AU)

Correspondence Address: JONES DAY, 222 EAST 41ST ST, NEW YORK, NY, 10017, US

(73) Assignee: Continence Control Systems International Pty.Ltd., Chastswood, New South Wales (AU)

(21) Appl. No.: 12/083,440

(22) PCT Filed: Oct. 16, 2006
(86) PCT No.: PCT/AU2006/001514

§ 371 Date: Mar. 16, 2010

(30) Foreign Application Priority Data
Oct. 14, 2005 (AU) 20055905672

(51) Int. Cl. A61M 1/12; A61N 1/362

(52) U.S. Cl. 600/17; 600/16

(57) ABSTRACT

The present invention relates to a method and apparatus for the treatment of a heart condition such as angina and/or congestive heart failure. Conditions such as angina, caused by inadequate blood flow to the coronary arteries, are treated in this invention by implantation of smooth muscle tissue wrapped around the ascending aorta. The smooth muscle tissue is stimulated electrically, by the implanted stimulator, to contract at timed intervals relative to the hearts' cycle. The contraction assists blood flow to the coronary vessels.

U.S. Pat. No. 6,659,936 issued on 9 Dec. 2003, International Patent Application PCT/AU00/00925 filed on 4 Aug. 2000, Australian Provisional Application AU PQ2026, filed on 4 Aug. 1999, relate to the control of continence.

International Patent Application PCT/AU2005/001698 filed on 8 Nov. 2005, Australian Provisional Application AU2004906393, filed on 8 Nov. 2004, relate to an implantable electrode arrangement.

International Patent Application PCT/AU2006/001301 filed on 4 Sep. 2006, Australian Professional Application AU2005904830, filed on 2 Sep. 2005, relate to an implant for managing a medical condition.

Australian Provisional Application No:2005905672 relates to a method and apparatus for treating a heart condition.

Each one of the above documents are incorporated herein by reference in their entirety.

### FIELD OF THE INVENTION

The present invention relates to a method and apparatus for the treatment of a heart condition, and, particularly, but not exclusively, to a method, apparatus and system for management of angina and/or congestive heart failure.

### BACKGROUND OF THE INVENTION

Angina is a condition caused by inadequate blood flow to the coronary arteries of the heart itself. It is usually associated with atherosclerosis of the arteries. It is a serious condition in itself and affects many people. It can severely affect lifestyle even in its “stable” form, preventing suffers from taking part in vigorous exercise or activity, for example. “Unstable” angina can be even more dangerous, as it is unpredictable.

Further, if angina is left untreated, it can lead to weakening of the heart and congestive heart failure as the efficiency of the pumping action of the heart deteriorates.

A number of treatments are available for addressing angina and symptoms of a weakened heart. These include:
Left Ventricular Assist Devices (LVAD). LVAD's are essentially pumps that assist or supplement the operation of the left ventricle. They supplement the performance of a weakened heart and facilitate perfusion to the rest of the body. They may not completely alleviate the condition of inadequate coronary blood flow (if it exists), and rather assist blood flow around the body generally. They are also quite large devices which generally require a large power supply and are, therefore, restrictive to wear.
Angioplasty is utilised to treat atherosclerosis, by introducing a catheter to break up plaque with or without the addition of coronary artery stents. Angioplasty may have limited affect, particularly in severe cases.
A technique called aortomyoplasty has been published. This utilises skeletal muscle (preferably from the latissimus dorsi) wrapped about the aorta. The skeletal muscle is electrically stimulated to produce a diastolic counter pulsation, in order to facilitate flow from the aorta. A problem with the use of skeletal muscle is that a vascularized graft is required: with complex and involved surgery to keep the nerves and blood supply intact. Further, because skeletal muscle must be taken from elsewhere in the patient's body, this can have a functional impact on the patient. Skeletal muscle also requires a relatively high frequency of electrical stimulation to adequately activate the muscle to generate force, and a large power source is required.

Other treatments that provide a counter pulsation effect include intra-aortic balloon pumps (utilizing an intra-aortic catheter that is temporarily introduced percutaneously in the patient and operated at the bedside in a hospital environment), Enhanced External Counter-Pulsation techniques (use of inflatable cuffs placed on the limbs with the patient lying in a bed) and Wearable Counter-Pulsation systems (utilising an inflatable cuff around the aorta). None of these or the above techniques are wholly effective, or applicable to treat all patients.

In International Patent Application No. PCT/AU00/0095 (referred to above), a method and apparatus is proposed for treating urinary incontinence which includes the steps of forming a “neosphincter” from smooth muscle tissue taken from elsewhere in the patient's body, and wrapping the neosphincter around the urethra. An implantable stimulator provides an electrical signal to the neosphincter via an electrode or electrodes. The electrical signal stimulates the neosphincter to maintain tone about the urethra to reduce leaks from the bladder until the user wishes to urinate. A signal from a control device may cause the stimulator to stop providing the electrical signal to the neosphincter, to allow the neosphincter to relax and enable the individual to urinate.

### SUMMARY OF THE INVENTION

In accordance with a first aspect, the present invention provides an apparatus for treating a heart condition, including a stimulator arranged to provide a signal to stimulate contractile tissue positioned at the heart or at a heart blood vessel, whereby to cause the contractile tissue to contract to assist heart function.

In an embodiment, the stimulator is arranged to be implanted within a patient. In an embodiment, the entire stimulator may be implanted within a patient. In another embodiment, a part of the stimulator may be implanted in the patient, and a part external.

In an embodiment, the stimulator may be external to the patient and provide stimulation signals across the skin to stimulate the contractile tissue. The contractile tissue may be positioned proximate to the heart or on the heart.

It may be positioned proximate to a heart blood vessel or on a heart blood vessel. The term “heart blood vessel” includes the blood vessels that enter and exit the heart, including the superior vena cava, the aorta, and also the coronary blood vessels or any other blood vessels associated with supply of nutrients and removal of waste products from the heart.

In an embodiment, the contractile tissue is positioned about the ascending aorta. In this embodiment, the contractile tissue is preferably implanted as a “wrap” about the ascending aorta. It may extend partly around the aorta or all around the aorta.

In this embodiment the contractile tissue is positioned prior to the coronary arteries so that its contraction may assist perfusion to the coronary blood vessels.

In an embodiment, the stimulator is arranged to provide a signal at a predetermined time relative to the cardiac cycle in order to stimulate the contractile tissue at a predetermined period relative to contraction of the heart.

In an embodiment, the timing of the stimulation is such as to cause contraction to provide a counter-pulsation effect during the latter phase of ejection of blood from the left ventricle.

In an embodiment, the stimulator includes a sensor for sensing phase of the cardiac cycle. This can be used to sense, for example, when the right ventricle contracts and this information may be used to time the signal to the contractile tissue.

In an embodiment, the contractile tissue is any contractile tissue apart from skeletal muscle tissue.

In an embodiment, the contractile tissue has properties the same or similar to those of smooth muscle.

In an embodiment, the contractile tissue is smooth muscle tissue. Smooth muscle tissue may be implanted smooth muscle tissue from another part of the patient's body, may be donated smooth muscle tissue, may be smooth muscle tissue grown from smooth muscle stem cells or proliferative smooth muscle cells, or may be a combination of grown smooth muscle tissue and/or donated smooth muscle tissue.

The use of smooth muscle as the contractile tissue gives rise to advantages such as;
smooth muscle implants can be implanted using a free graft, using a suitable smooth muscle source (vein or artery) in a convenient location to the aorta;
no microsurgery is required to preserve the viability of the donor tissue (compared with skeletal muscle in which a tethered graft or microsurgery is required to keep nerve and blood supply intact);
there is little or no functional impact on the patient (compared with skeletal muscle which impacts when a skeletal muscle implant is taken from elsewhere in the body);
smooth muscle does not require high frequency electrical stimulation to activate the muscle (as compared with skeletal muscle) so there is an advantage that a smaller power source is required for the stimulator.

In an embodiment, the contractile tissue may be placed at another position at the heart or a heart blood vessel. For example, it may be placed about the descending aorta and still may achieve a counter pulsation effect.

In accordance with a second aspect, the present invention provides a device for treating a heart condition, the device including contractile tissue implanted at the heart or at a heart blood vessel, the contractile tissue being arranged to be stimulated to contract, whereby to assist heart function.

In an embodiment, the contractile tissue is placed about the ascending aorta.

In an embodiment, the contractile tissue is placed as a wrap about the ascending aorta. The contractile tissue may be placed prior to the coronary arteries, in order to assist perfusion to the coronary blood vessels. This is of advantage in treating conditions such as angina.

In an embodiment, the contractile tissue is smooth muscle tissue.

In accordance with a third aspect, the present invention provides a programmer for programming operation of a stimulator arranged to provide a signal to cause contractile tissue to contract to assist heart function, the programmer including an interface enabling communication with the stimulator for programming of the implantable stimulator.

In an embodiment, the programmer may be utilised to set stimulation signal parameters of the stimulator.

The programmer may include a telemetry arrangement enabling communication with the stimulator, which may, in an embodiment, include a communicator for communicating data on stimulator operation to the programmer. The programmer may, for example, be used post-operatively by a cardiologist to adjust operating parameters of the stimulator.

In accordance with a fourth aspect, the present invention provides a system for treating a heart condition, the system including a stimulator in accordance with the first aspect of the invention and a device in accordance with the second aspect of the invention.

In an embodiment, the system may also include a programmer in accordance with the third aspect of the invention.

In accordance with a fifth aspect, the present invention provides a system for treating a heart condition, including a stimulator in accordance with the first aspect of the invention, and a programmer in accordance with the third aspect of the invention.

In accordance with a sixth aspect, the present invention provides a method of treating a heart condition, comprising the steps of stimulating contractile tissue positioned at the heart or at a heart blood vessel, whereby to cause contractile tissue to contract to assist heart function.

In an embodiment, the contractile tissue is smooth muscle tissue.

In an embodiment, stimulation is timed with respect to the cardiac cycle. In one embodiment the stimulation is timed to provide contraction of the contractile tissue to provide a counter-pulsation effect.

In an embodiment, the contractile tissue is positioned about the ascending aorta prior to the coronary arteries, so that its contraction (timed appropriately in relation to the cardiac cycle) facilitates perfusion to the coronary blood vessels. This embodiment has advantages in the treatment of heart conditions such as angina.

In accordance with a seventh aspect, the present invention provides a method of treating a heart condition, comprising the step of implanting into a patient an implantable stimulator arranged to provide stimulation signals to contractile tissue in order to cause the tissue to contract to assist heart function.

In accordance with an eighth aspect, the present invention provides a method of treating a heart condition, comprising the steps of implanting contractile tissue at a position at the heart or at a heart blood vessel, the tissue being arranged to be stimulated to contract and assist heart function.

In an embodiment, the contractile tissue is smooth muscle tissue.

### BRIEF DESCRIPTION OF THE DRAWINGS

Features and advantages of the present invention will become apparent from the following description of an embodiment thereof, by way of example only, with reference to the accompanying drawings, in which;

FIG. 1 is a schematic diagram illustrating a system and apparatus in accordance with an embodiment of the present invention, illustrated together with a diagram of a heart;

FIG. 2 is a Wiggers diagram for the left side of a heart;

FIG. 3 is a block diagram of componentry of an implantable stimulator of the system of FIG. 1;

FIG. 4 is a block diagram of an embodiment a system in accordance with the present invention;

FIG. 5 is a schematic diagram illustrating an apparatus in accordance with a further embodiment of the present invention;

FIGS. 6, 7 and 8 are exploded perspective, plan and side views, respectively, of an electrode arrangement for delivering stimulation signals in an apparatus in accordance with an embodiment of the present invention;

FIGS. 9, 10, 11 and 12 are perspective, plan, side section and detailed views of a shroud component of the electrode arrangement of FIG. 6, and

FIGS. 14, 15, 16, 17, 18 are perspective, rear, plan section, side section and plan views of a cover component of the electrode arrangement of FIG. 6.

### DETAILED DESCRIPTION OF THE PREFERRED EMBODIMENT

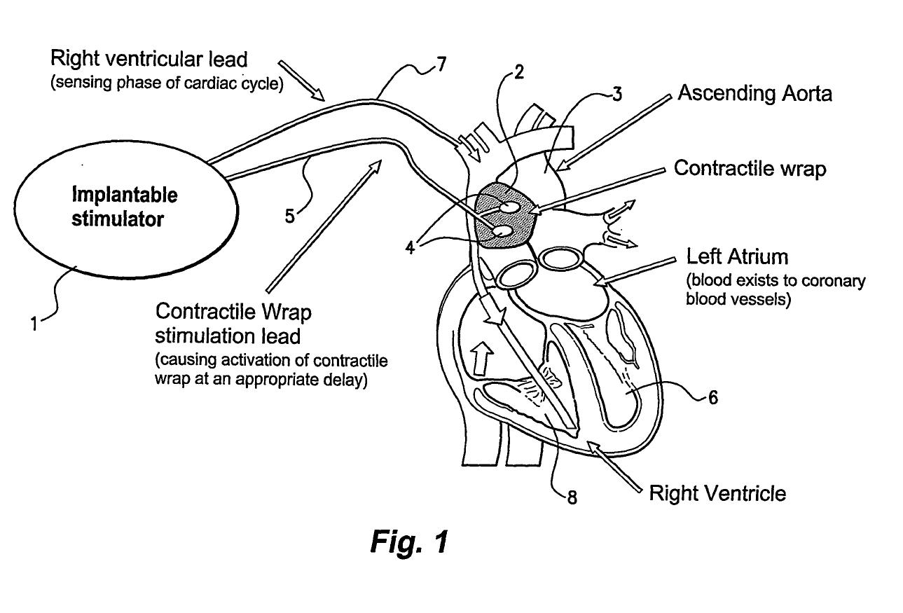

Referring to FIG. 1, a system and in accordance with an embodiment of the present invention, for treating a heart condition, is shown in schematic form. The system includes an apparatus comprising an implantable stimulator 1 which is arranged to provide stimulation signals to contractile tissue 2 which is, in this example, is placed as a wrap about the ascending aorta 3. Electrodes 4 at the contractile tissue 2 are conductively connected to the stimulator in order to transmit the signal from the stimulator 1 to the contractile tissue 2. A conductive lead 5 connects the stimulator 1 to the electrodes 4.

In operation, the contractile tissue 2 is caused to contract periodically to assist heart function. In this particular embodiment, the contractile wrap 2 is placed about the ascending aorta 3 before the blood flow reaches the coronary arteries. The stimulator 1 is timed to provide a signal to the contractile wrap 2 so that it contracts in order to provide a counter-pulsation effect during the latter phase of ejection of blood from the left ventricle 6. The contraction results in an increase in perfusion to the coronary blood vessels distal to the contractile wrap 2. It also has the beneficial effect of increasing blood flow peripherally by additional emptying of the ascending aorta 3.

In order to facilitate correct timing of the signal to the contractile wrap 2, a conductive lead 7 is connected between the right ventricle 8 and the implantable stimulator 1. This right ventricular lead 7 includes an electrode placed in the right ventricle 8 and provides signals back to the implantable stimulator 1 which includes a control unit 9 (to be described later) which is able to determine from the sensor signals when electrical activation of the right ventricle 8 is occurring. At a predetermined delay after right ventricular 8 electrical activation, the stimulator provides the stimulation signal to the contractile wrap 2, to provide the counter-pulsation effect.

In this embodiment, the contractile tissue is smooth muscle tissue. The smooth muscle tissue may be obtained from elsewhere in the body, formed into a wrap and surgically implanted about the ascending aorta 3. Alternatively, the smooth muscle tissue may be grown from smooth muscle stem cells and/or proliferative smooth muscle cells. Alternatively, the smooth muscle tissue may be transplanted smooth muscle tissue augmented by smooth muscle stem cells and/or proliferative smooth muscle cells.

International Patent Application number PCT/2006/001301, referred to above, disclose augmentation of contractile tissue using proliferative smooth muscle cells, smooth muscle stem cells, growth factors (trophic and/or neurotrophic).

Smooth muscle may be taken from anywhere or grown (as discussed above). In an embodiment, the smooth muscle may be taken from the smooth muscle of the bladder and transplanted around the ascending aorta, with its circulation intact. Alternatively, the muscle is venous smooth muscle, artery or arteriole, anococcygeus smooth muscle or terminal ilium transplanted as a segment devoid of mucosa and having its circulation intact. A further alternative is the dartos smooth muscle from the scrotum or a portion of the vagina or the labia.

In an embodiment, smooth muscle may be taken as a free graft. In this case the tissue is separated from its normal circulation and becomes vascularised by ingrowth of blood vessels at the site of implant.

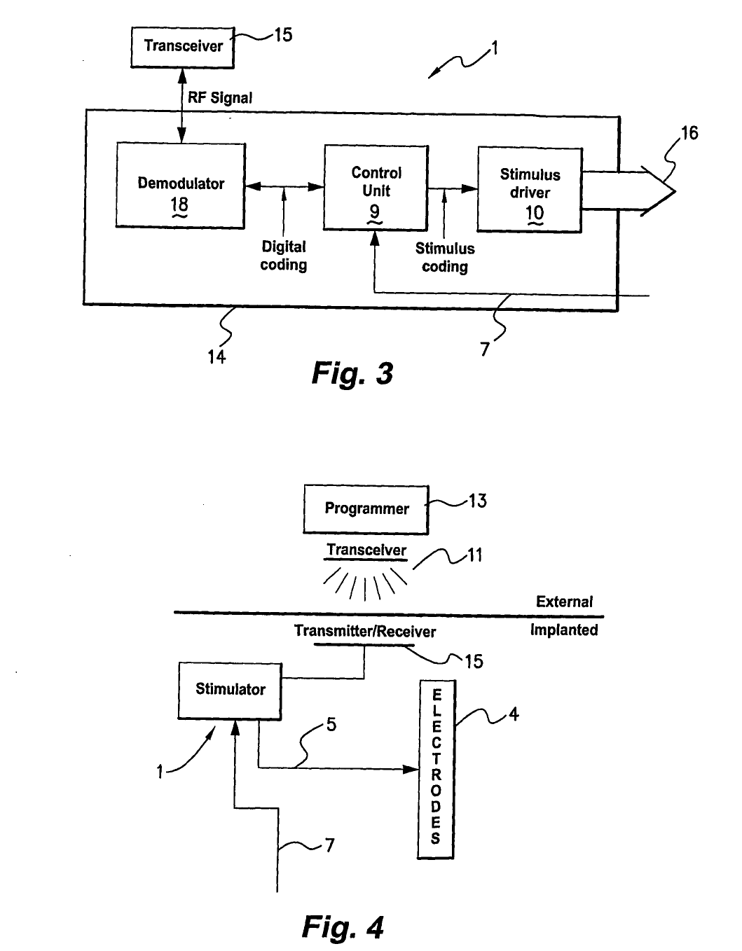

The stimulator 1 is shown in more detail in FIG. 3. In this embodiment, a signal generator means arranged to provide an electrical signal for stimulation of the smooth muscle wrap 2 is in the form of a control unit 9 and stimulus driver 10. The control unit 9 encodes the stimulus and provides a signal to the stimulus driver 10 which provides the stimulation signal at output 16. The output 16 outputs to conductor 5 and to one or more electrodes 4. The implantable stimulator may be placed in subclavicular region (similar to placement of heart pacemakers). It could be implanted in other places, however.

The control unit 9 also receives input from the right ventricular lead 7, for detecting the right ventricular electrical activation. The control unit 9 utilises the detected signal to calculate the timing of the signal to the contractile tissue 2.

In this embodiment, the control unit 9 and stimulus driver 10 form, together with a demodulator 18, a processing unit for generating the stimulation signal(s) at output 16.

The demodulator 18 is arranged to demodulate a signal received by transceiver 15. An external programmer unit 13 is able to communicate via the transceiver 15 with the processing unit 14 in order to adjust parameters of the stimulator 1. In addition, as described in more detail later, the processing unit 14 may transmit, via control unit 9, demodulator 18 and transceiver 15, signals to the programmer unit. The transmitted signals may deliver telemetry information indicative of parameters of the stimulator, for the purposes of calibration and control.

The entire stimulator 1 (including components 14 and 15), is enclosed in a housing which includes a casing made from a bio-compatible material, such as titanium, silicone polymer or other acceptable materials or combinations of materials, including, but not limited to inert materials. Metallic electrodes and leads may be of platinum-iridium alloy. The connecting wires are, in an embodiment, insulated with a silicone coating. The frequency of the electromagnetic signal (typically at a radio frequency (RF)) for transmission and reception by the transceiver 15 may depend on the material of the casing of the stimulator.

The stimulation signal 16 provided to contract the smooth muscle wrap is selected so as to provide a substantial increase in tone in the wrap and to squeeze blood from the ascending aorta. A generally rectangular and symmetrically biphasic pulse may be suitable for this. The signal has a substantially constant current less than or equal to 50 mA, 15 mA, 10 mA, or 5 mA, and in some preferred embodiments may be in the order of 4 mA, 8 mA, 12 mA, or 15 mA.

Stimulation pulse frequency provided to the wrap is in the range of 0.1 Hz to 5 Hz, 0.2 Hz to 4.0 Hz. 0.25 Hz to 3.0 Hz, 1 Hz to 3.0 Hz, 1.5 Hz to 3 Hz, 1.75 Hz to 2.5 Hz, or a 0.25 Hz to 2.25 Hz, and in one embodiment, is 1 Hz, 2 Hz, 2.5 Hz or 3 Hz.

Stimulation phase width of each phase is in the range of 0.05 ms to 2.0 ms, 0.1 ms to 1.5 ms, 0.2 ms to 1 ms, 0.25 ms to 0.75 ms, and in one embodiment is 0.2 ms, 0.4 ms, 0.5 ms or 1 ms. The stimulator is current regulated, and accordingly the stimulation voltage will vary with the resistance of the muscle tissue between the electrodes.

It is also possible to use a burst of stimulation (for example at 5 or more Hz) to initiate activation of the wrap which can then be applied on every second, or third, or more, heart beat to minimize the time required for the smooth muscle to generate force.

Typical values for the voltage are between 0.1 and 15 Volts, 0.2 and 12 Volts, 0.5 and 12 Volts, 0.5 and 10 Volts, or 0.5 and 7.5 Volts. In one embodiment, the voltage is 2.5 Volts, 5 Volts, 7.5 Volts or 10 Volts. Either a current source (voltage limited) or a voltage source (current limited) stimulator may be used.

It is also possible to use an asymmetric biphasic pulse, in which, for example, the first phase is shorter in duration than the second phase.

Following this activation, the stimulation to the smooth muscle wrap is then modified such that the wrap relaxes before an appropriate electrical cardiac synchronisation event is detected once more.

The smooth muscle tissue may in one embodiment comprise an innervated vain or artery, which is able to contract within 300 to 400 ms and relax again in 200 to 300 ms. Depending on contractile tissue utilised, a signal may be need to be applied to the contractile tissue for a predetermined period to allow tension to build in the tissue to have the desired counter-pulsation effect. With smooth muscle, 3000 to 300 ms may be necessary to allow tension to build, in an embodiment 1000 to 400 ms, in an embodiment 550 to 450 ms, in another embodiment 500 ms.

FIG. 4 shows a system in accordance with an embodiment of the present invention, including a programmer unit 13 which may be utilised by a physician to set and adjust parameters of the implanted stimulator 1. The programmer unit 13 is arranged for communication with the stimulator via transceiver 11, and may include a computing device. The control unit 9 is also arranged to transmit stimulator telemetry information indicative of one or more of the parameters of the stimulator 1, for detection by the programmer 13 via transceiver 11. The programmer unit 13 can therefore determine parameters of the stimulator from telemetry information and can adjust the parameters by transmitting control signals to the stimulator 1. The signal from the programmer may be able to selectively vary the output current, shape, frequency and/or pulse width of the stimulation signal(s) and the delay from electrical detection of ventricular activation to stimulation of the smooth muscle wrap and any other such stimulation and/or timing parameters to provide an effective counter pulsation effect using a smooth muscle wrap.

In operation, a physician adjusts parameters of the stimulation signal(s). The physician will note feedback from the patient as to the effect of the stimulus and may subsequently re-adjust the parameters until the stimulation is optimum. For example, patient perceived feedback may be used to set the maximum stimulation threshold of the smooth muscle wrap 2.

In the above-described embodiments, signals between the programmer and the stimulator are RF signals. Other types of transmission media other than RF may be used. For example, microwave signals may be used for transmission, optical signals may be used.

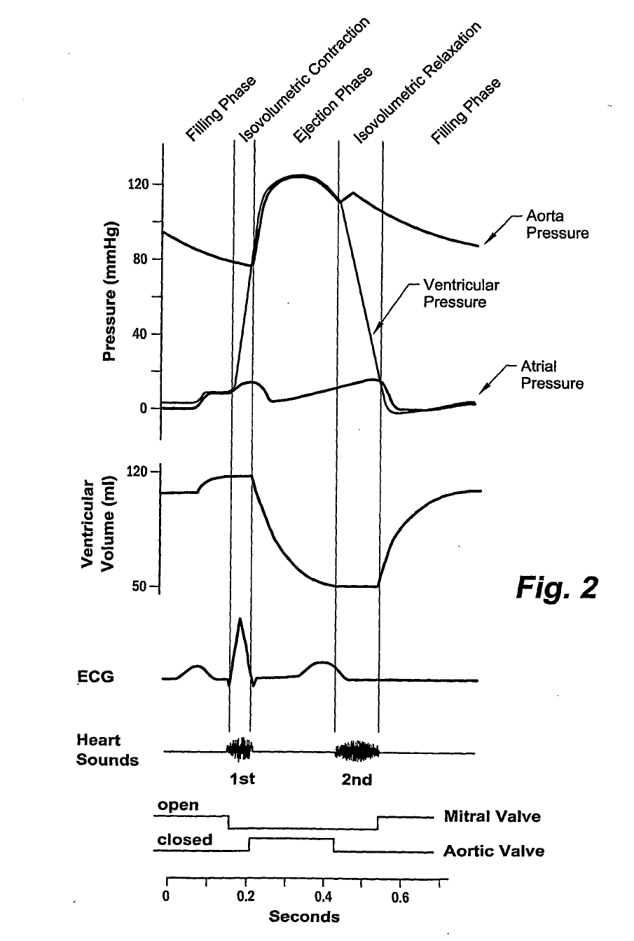

The Wiggers diagram shown in FIG. 2 shows key electrical and mechanical events in contraction of the mammalian heart. The peak ventricular ejection phase occurs approximately 200-250 ms after electrical activation of the ventricle (indicated by the Q wave electrocardiogram). As discussed above, some types of tissue, in particular smooth muscle tissue, may require longer periods of stimulation to enable the appropriate tension to build. In some cases, therefore, depending on the tissue, a stimulation signal may be provided to the tissue before electrical activation of the ventricle. In this case, electrical activation of the ventricle is sensed and for the next cycle of signal stimulation, signal stimulation is applied before the next anticipated electrical activation of the ventricle. As part of the timing cycle of the system, the smooth muscle wrap may only be activated 10 to 30 times per minute, to prevent it forming an impediment to the ventricular ejection phase. It will be appreciated that the invention is not limited to activating the smooth muscle wrap 10 to 30 times per minute. It may be activated more or less than this, if clinically appropriate.

In the above embodiment, a right ventricular intravascularly placed electrode is used to sense ventricular electrical activation, in order to time the signal to the contractile tissue. The present invention is not limited to this sensing. Alternatively, an epimyocardial electrode may be placed on the surface of the right (or left) ventricle to detect the underlying electrical activation and thus synchronise the counter-pulsation. More than one sensing electrode may be utilised in either the right ventricular or left ventricular arrangement. Other arrangements for sensing may be used.

In the above embodiments, any suitable electrode(s) may be utilised to stimulate the implant 2. For example, button electrodes, cuff electrodes or any other suitable electrode(s) may be utilised.

In embodiments, an electrode arrangement such as a disclosed in PCT/AU/20054/001698 may be utilised.

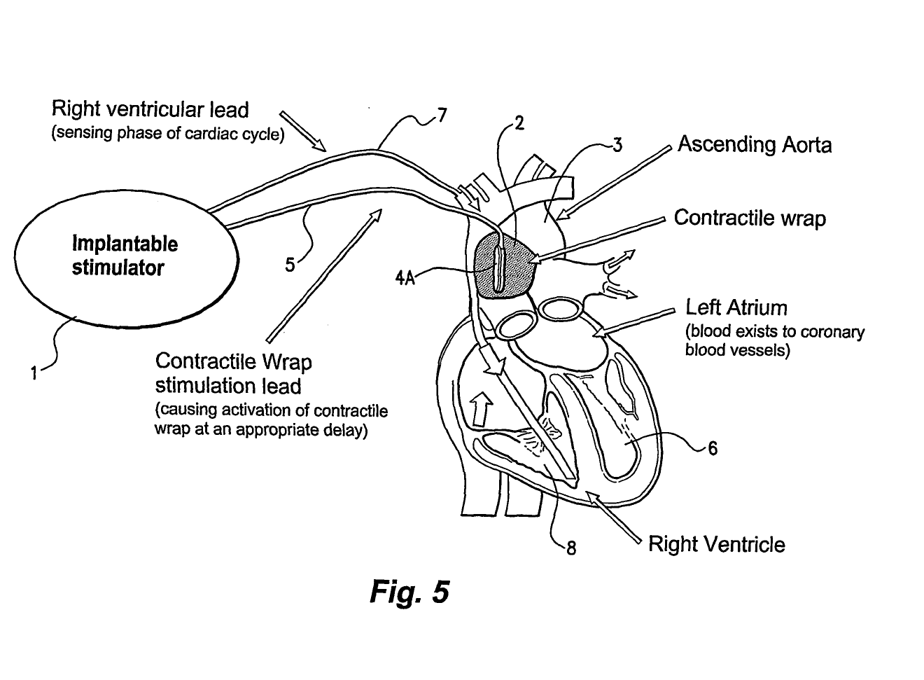

FIG. 5 illustrates an embodiment of the present invention where a “peg” electrode 4A such as disclosed in PCT/AU20054/001698 is utilised to transmit signals to the implant 2 from the stimulator 1.

In FIG. 5, the same reference numerals as used in previous embodiments have been utilised to designate similar components, and no further description will be given here of these components.

The electrode 4A will now be described in more detail.

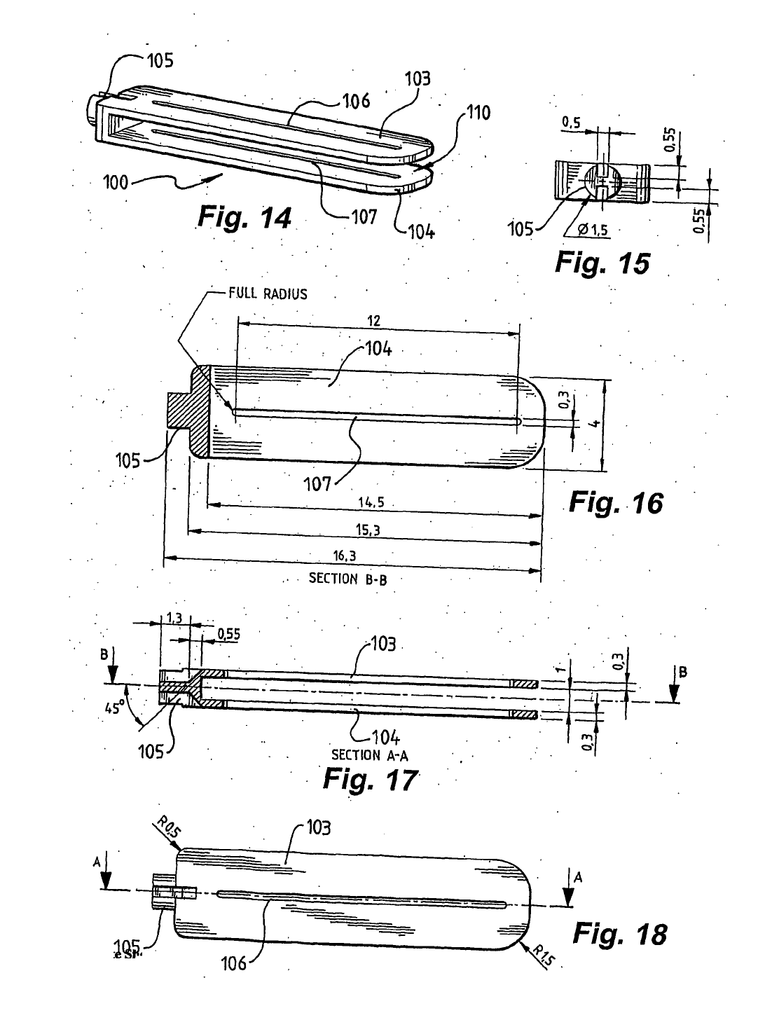

The electrode comprises a number of components. These include an electrode cover 100 (shown in most detail in FIGS. 13 through 17).

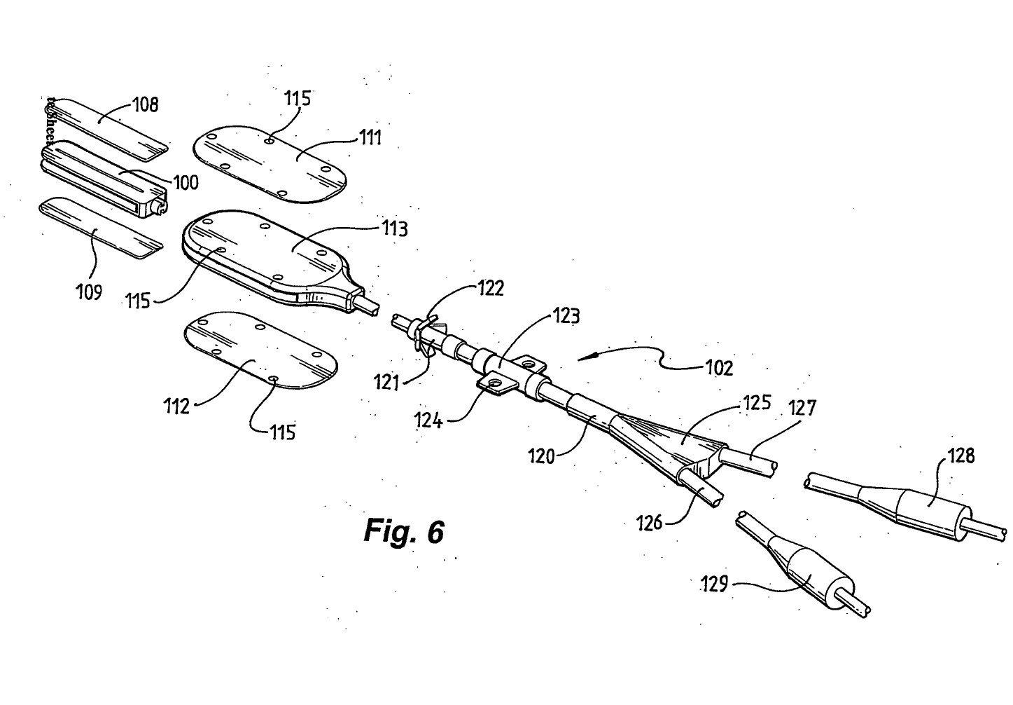

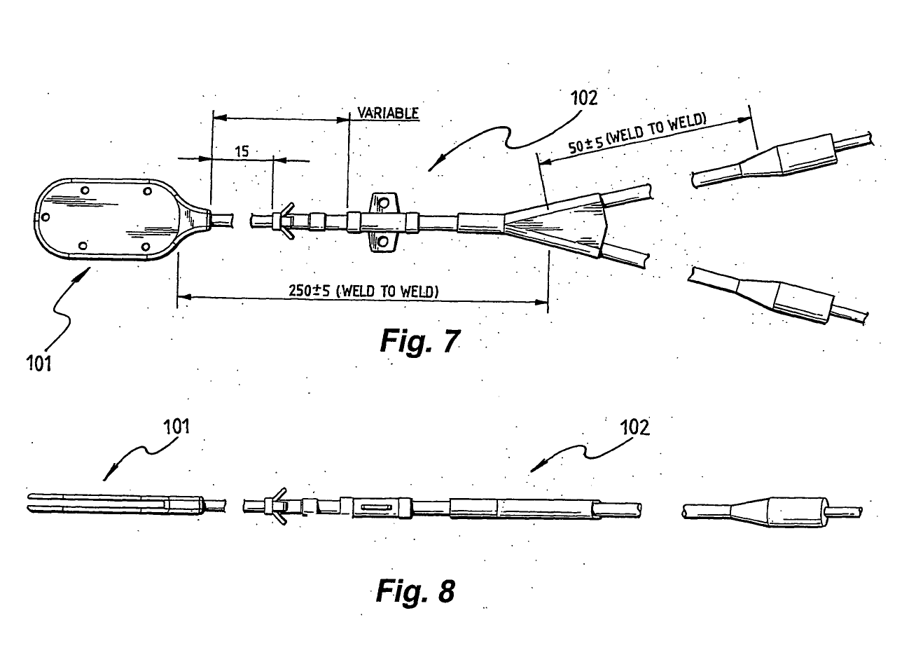

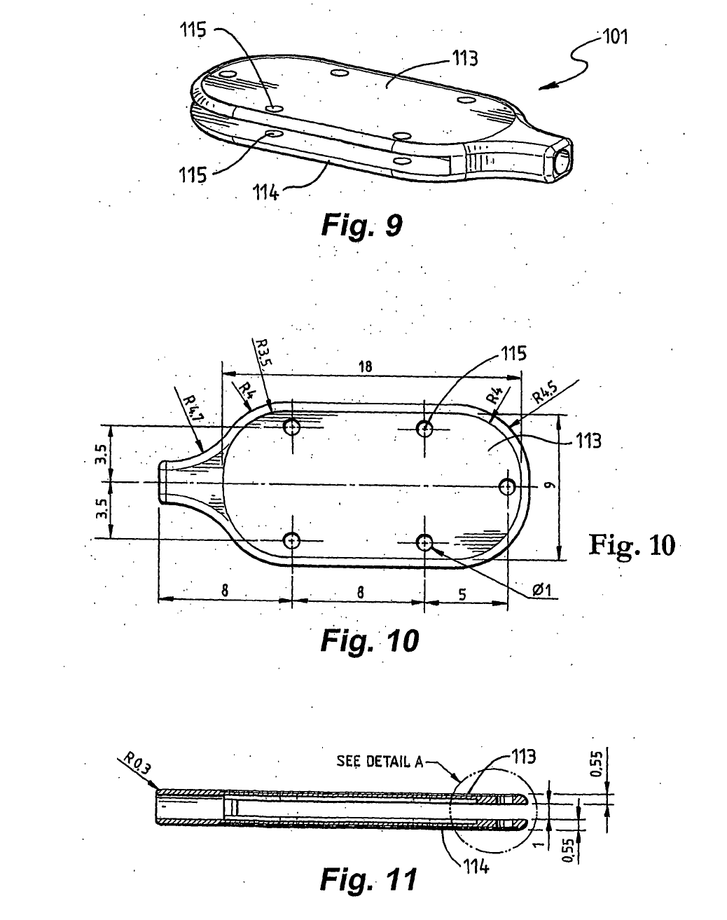

The components also include an electrode shroud (shown in best detail in FIGS. 9 through 12) and also an electrode lead 102 (shown in FIGS. 6, 7 & 8, together with the other components of the electrode arrangement).

In this embodiment first and second electrode elements are formed by the electrode cover 100, which includes insulating elements 103,104 extending from a base 105. The insulating extending elements 103,104 are formed with a slot 106,107, respectively, extending substantially along the length of the extending elements 103,104. When the electrode arrangement is assembled, platinum foil electrodes 108,109 (FIG. 6) are placed on the outer surfaces of the elements 103,104 so that they are insulated from the gap 110 formed between the elements 103,104 apart from the slots 106,107, which expose portions of the conductive plates 108,109 to the gap 110 (and, in use, to any tissue seated within the gap).

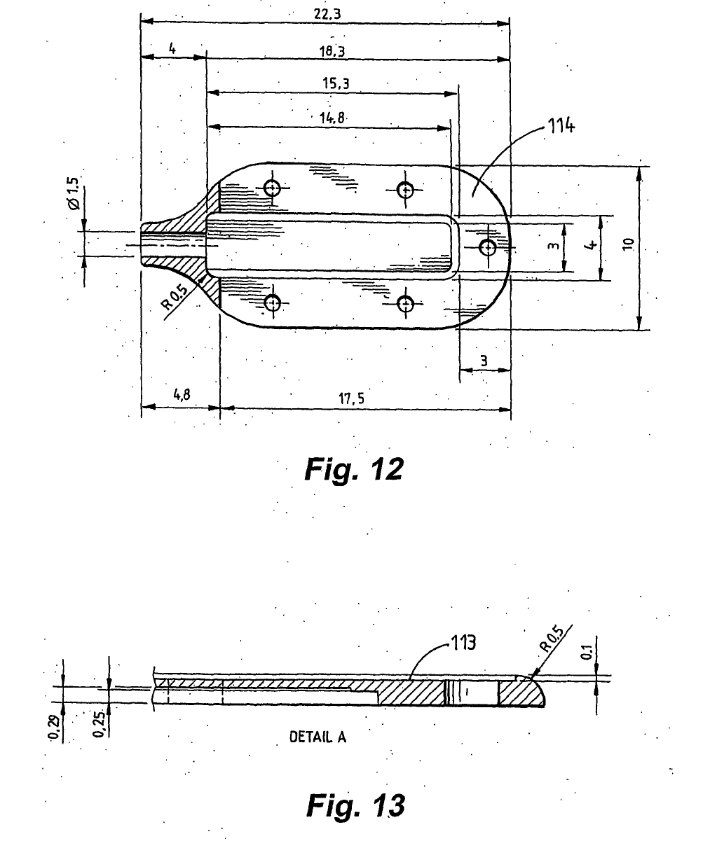

When assembled, the electrode cover 100 and platinum electrode foils 108,109 seat within the electrode shroud 101 as best shown in FIGS. 9, 10, 11 & 12. FIG. 12 in particular shown in cross-section where the electrode cover seats.

Electrode shroud 101 is formed from silicone. In order to provide reinforcement, PET mesh covers 111,112 are provided to fit to upper 113 and lower 114 extending portions of the shroud 101. Suture holes 115,116 are provided in the covers 111,112 and also in the elements 113,114 of the shroud 101. Note that the reinforcement can be provided by other means and is not limited to PET mesh. Further, the electrode shroud need not be in silicone but could be of other bio-compatible material and may not require re-inforcement. Further, note that other means for affixing to the tissue may be provided other than suture holes or instead of suture holes.

The electrode lead 102 is a multi-component arrangement which includes an outer insulating cover 120, a tine collar 121 including tines 122 for retaining the lead in position within a patient. It also includes a sutured collar 123 including suture holes 124 for suturing to patient tissue to also facilitate retaining the lead 102 in position. There is also bifurcation moulding 125 which enables the lead to split into two parts 126,127 which may contain separate conductors, and connectors 128,129 which may be arranged to contact to a simulation device.

In the above embodiments, the electrode arrangement includes a pair of electrode elements which extend away from a base which joins them together at their proximal ends. In a further embodiment, a single electrode element which is not joined at any base is provided. This single electrode element may be used to provide stimulation to contractile tissue on its own, or may be used together with one or more similar electrode elements to provide stimulation.

In the above described embodiments, each electrode element is provided with a single electrode. The single electrode is an elongate electrode extending substantially the majority of the length of the electrode element.

One advantage of having thin electrodes bounded by insulating material on either side is that the arrangement operates to confine the electric field produced by the electrode to the tissue immediately adjacent the electrode. This reduces or prevents stimulation of tissue that it is not desirable to stimulate eg. tissue external to a contractile tissue sphincter being controlled.

In operation, the electrodes 108, 109 and extending elements 103, 104 are positioned either side of the smooth muscle implant to enable signals to be transmitted to the implant for operation.

Electrode arrangement 4A allows application of an electric field between the opposing electrode elements to stimulate the tissue between them. The electric field in one embodiment is confined so that stimulation is to a band of tissue between the electrodes.

In one embodiment, innervation runs within the implant 2 perpendicular to the band of tissue being stimulated.

The elements in electrode 4A extend over the tissue in a manner analogous to that of a clothes peg.

Other electrode patterns then a single line electrode on the surfaces of the elements may be utilised.

In the above embodiment, the stimulator is a totally implantable device. In an alternative embodiment, the stimulator may not be implantable. The stimulator in this embodiment may comprise a stimulator device having similar componentry to that discussed above in relation to the embodiment of FIGS. 1 to 4, but being arranged to be placed externally of the patient. In one embodiment, signals are coupled to electrodes placed within the patient in order to stimulate the contractile tissue.

Coupling may be by way of inductively coupling the signals across the patient's skin to an internally positioned electrode arrangement. In another embodiment, part of the stimulator componentry may be placed outside the patient and part inside the patient.

In the above embodiment, the wrap is placed about the ascending aorta. It may be placed in other positions at the heart or at the heart blood vessels and is not limited to being placed at the ascending aorta. It may be placed at the descending aorta, for example.

In the above embodiment, the wrap completely encircles the aorta. In other embodiments, the wrap may extend only part way about a blood vessel such as the aorta. The wrap may be formed in any number of mechanical configurations. As discussed above, it may be formed all the way round the heart and, in one embodiment, have ends that meet and are secured together. In another embodiment, the wrap has overlapping ends which are then secured together. In yet another embodiment, the wrap may not extend entirely around the heart, but only part way round the heart. Other mechanical configurations may also be implemented. In yet another embodiment, the contractile tissue may be formed into a sling arrangement, which extends around the ascending aorta and which has ends secured to a convenient anatomical feature such as intercostal muscle, bone, or another area of facia. Contraction of the tissue will create an indent in the ascending aorta having a similar counter pulsation effect to a wrap.

In the embodiment of FIG. 1. a pair of electrodes is shown for stimulation of contractile tissue. The invention is not limited to a pair of electrodes. A single electrode or more than two electrodes may be used.

In above embodiments, a single stimulation signal generator is used to provide the electrical signal. The invention is not limited to a single generator. Other embodiments may use two or more signal generators.

Other embodiments may use two or more stimulators, which may be placed in different locations.

In the above embodiment, the system includes a programmer unit. It is feasible that in some cases a programmer unit may not be required.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

The claims defining the invention are as follows:

1. An apparatus for treating a heart condition, including an implantable stimulator arranged to provide a signal to stimulate contractile tissue positioned at the heart or at a heart blood vessel, whereby to cause the contractile tissue to contract to assist heart function.

2. An apparatus in accordance with claim 1, wherein the contractile tissue is positioned about the ascending aorta.

3. An apparatus in accordance with claim 2, wherein the contractile tissue is formed as a wrap about the ascending aorta.

4. An apparatus in accordance with claim 2 or claim 3, wherein the contractile tissue is positioned between the heart and coronary artery, whereby its contraction assists perfusion to the coronary blood vessels.

5. An apparatus in accordance with any one of the preceding claims, wherein the stimulator is arranged to provide the signal to cause the contractile tissue to contract at a predetermined time relative to contraction of the heart.

6. An apparatus in accordance with claim 5, wherein the timing of the provision of the signal is such as to cause contraction of the contractile tissue to provide a counter-pulsation effect during the latter phase of ejection of blood from the left ventricle.

7. An apparatus in accordance with any one of the preceding claims, including means for sensing information on the phase of the cardiac cycle.

8. An apparatus in accordance with claim 7, wherein the sensed information is used to time the signal to the contractile tissue.

9. An apparatus in accordance with any one of the preceding claims, wherein the contractile tissue is smooth muscle tissue.

10. A device for treating a heart condition, the device including contractile tissue implanted at the heart or at a heart blood vessel, the contractile tissue being arranged to be stimulated to contract, whereby to assist heart function.

11. A device in accordance with claim 10, wherein the contractile tissue is implanted about the ascending aorta.

12. A device in accordance with claim 11, wherein the contractile tissue is formed into a wrap about the ascending aorta.

13. A device in accordance with claim 11 or claim 12, wherein the contractile tissue is placed prior to the coronary artery, whereby to assist perfusion to the coronary blood vessels.

14. A device in accordance with any one of claims 10 to 13, wherein the contractile tissue is smooth muscle tissue.

15. A programmer for programming operation of an implantable stimulator arranged to provide a signal to cause contractile tissue to contract to assist heart function, the programmer including an interface enabling communication with the implantable stimulator for programming of the implantable stimulator.

16. A programmer in accordance with claim 15, the interface enabling stimulation signal parameters of the implantable stimulator to be set.

17. A system for treating a heart condition, including an implantable stimulator in accordance with any one of claims 1 to 9 and a device in accordance with any one of claims 10 to 14.

18. A system in accordance with claim 17, further including a programmer in accordance with claim 15 or claim 16.

19. A system for treating a heart condition, including an implantable stimulator in accordance with any one of claims 1 to 9 and a programmer in accordance with claim 15 or claim 16.

20. A method of treating a heart condition, comprising the steps of stimulating contractile tissue positioned at the heart or at a heart blood vessel, whereby to cause the contractile tissue to contract to assist heart function.

21. A method in accordance with claim 20, wherein the contractile tissue is smooth muscle tissue.

22. A method in accordance with claim 20 or claim 21, wherein the stimulation is timed with respect to the cardiac cycle.

23. A method in accordance with claim 22, wherein the stimulation is timed to provide contraction of the contractile tissue to provide a counter-pulsation effect.

24. A method in accordance with any one of claims 20 to 23, wherein the contractile tissue is positioned about the ascending aorta prior to the coronary arteries, so that its contraction facilitates perfusion to the coronary blood vessels.

25. A method in accordance with any one of claims 20 to 24, wherein the contractile tissue is smooth muscle tissue.

26. A method of treating a heart condition, comprising the step of implanting into a patient an implantable stimulator arranged to provide stimulation signals to contractile tissue in order to cause the tissue to contract to assist heart function.

27. A method of treating a heart condition, comprising the steps of implanting contractile tissue at a position at the heart or at a heart blood vessel, the tissue being arranged to be stimulated to contract and assist heart function.
